(19) United States
(12) Patent Application Publication
(10) Pub. No.: US 2010/0069641 A1
(43) Pub. Date: Mar. 18, 2010

(54) PROCESS FOR THE PREPARATION OF SODIUM SALT OF 1-(((1(R)-(3-(2-(7-CHLORO-2-QUINOLINYL)-ETHENYL)PHENYL)-3-(2-(1-HYDROXY-1-METHYLETHYL)PHENYL)PROPYL)SULFANYL)METHYL)CYCLOPROPANEACETIC ACID

(75) Inventors: Daniel Zyla, Starogard Gdanski (PL); Robert Rynkiewicz, Starogard Gdanski (PL); Mariusz Krzyzanowski, Skorcz (PL); Jan Ramza, Pruszkow (PL)

Correspondence Address: MATTHIAS SCHOLL, 14781 MEMORIAL DRIVE, SUITE 1319, HOUSTON, TX, 77079, US

(73) Assignee: ZAKLADY FARMACEUTYCZNE POLPHARMA S.A., Starogard Gdanski (PL)

(21) Appl. No.: 12/597,746

(22) PCT Filed: Apr. 30, 2008
(86) PCT No.: PCT/PL08/00033

§ 371 Date: Oct. 26, 2009

(30) Foreign Application Priority Data
May 2, 2007 (PL) P.382346

(51) Int. Cl. C07D 215/18

(52) U.S. Cl. 546/172

(57) ABSTRACT

A novel process for the preparation of a sodium salt of 1-(((1(R)-(3-(2-(7-chloro-2-quinolinyl)-ethenyl)phenyl)-3-(2-(1-hydroxy-1-methyl-ethyl)phenyl)propyl)sulfanyl)methyl)cyclopropaneacetic acid, comprising: a) reacting a compound of Formula 1 with methanesulfonyl chloride in the presence of a tertiary amine to yield a crude solution of a compound of Formula 2; b) filtering the crude solution of compound of Formula 2 obtained in a) to remove solid amine hydrochloride, reacting the filtrate without isolation or further purification with a compound of Formula 3, and isolating 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid; c) reacting the 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid isolated in b) with tert-butylamine to yield a compound of formula 4; d) isolating and purifying the compound of formula 4; and e) converting the compound of formula 4 to a compound of formula 5.

### CROSS-REFERENCE TO RELATED APPLICATIONS

This is a National Stage Application of International Patent Application No. PCT/PL2008/000033, with an international filing date of Apr. 30, 2008, which is based on Polish Patent Application No. P.382346, filed May 2, 2007.

### BACKGROUND OF THE INVENTION

1. Field of the Invention

The subject matter of the invention is a novel method for the preparation of a sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid:

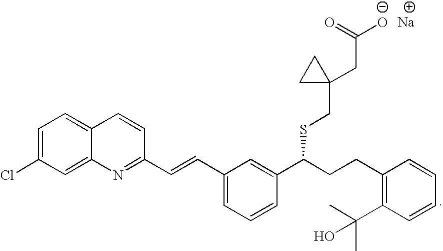

2. Description of the Related Art

1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid,

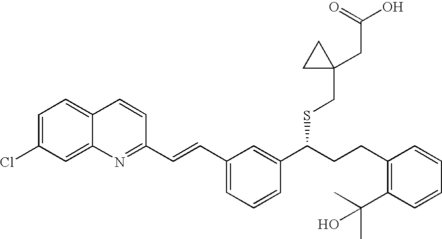

known under the international non-proprietary name (INN) of Montelukast, is indicated for the treatment of asthmatic and allergic diseases.

Montelukast-acid was disclosed in EP 0 480 717 B1.

Pharmaceutically acceptable salts of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid are described in Polish patent application P. 370850.

Conventional methods for the preparation of a sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid consist mainly in the preparation and isolation of crystalline 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid and its subsequent conversion into the sodium salt.

EP 0737186 B1 disclosed a method for the synthesis of the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid involving isolation of crystalline 2-(2-(3(S)-(3-(2-(7-chloro-2-quinolinyl)-ethenyl)phenyl)-3-(hydroxypropyl)phenyl)-2-propanol methanesulfonate. In EP 0737186 B1, a crystalline form of the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid was disclosed.

Another known method for the preparation of Montelukast sodium involves nucleophilic substitution reaction of the methanesulfonyl group in methanesulfonate using (1-(sulfanylmethyl)cyclopropyl)acetic acid dilithium salt. The dilithium salt is previously obtained through a reaction of two butyllithium equivalents with one equivalent of (1-(sulfanylmethyl)cyclopropyl)acetic acid, and methylsulfonate forms in the reaction of methanesulfonyl chloride with 2-(2-(3(S)-(3-(2-(7-chloro-2-quinolinyl)-ethenyl)phenyl)-3-(hydroxypropyl)phenyl)-2-propanol in the presence of diisopropylethylamine. To isolate the crystalline methanesulfonate, its slight solubility in acetonitrile is exploited, and the byproduct diisopropylethylamine hydrochloride remains dissolved in methanesulfonate filtrates. The reaction mixture is acidified and Montelukast is extracted into the solvent as an acid to be purified by recrystallization of the dicyclohexylamine salt thereof. The obtained salt is then converted back into the Montelukast-acid and the acid is converted subsequently into the crystalline sodium salt.

In WO 2006/043846 disclosed is the reaction of (1-(sulfanylmethyl)cyclopropyl)acetic acid disodium salt with methanesulfonate, wherein the disodium salt is formed using sodium tert-butanolate, and Montelukast is isolated as a crystalline tert-butylamine salt. The salt of Montelukast with tert-butylamine may be employed in the preparation of high-purity free 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid and/or sodium salt thereof. According to WO 2006/043846, the free 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is isolated in its crystalline form and may be recrystallized from a lower alcohol if needed.

WO 2005/074935 describes the polymorphic forms of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid.

The issues of the polymorphism of the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid were described in WO 2005/075427.

The 2-(2-(3(S)-(3-(2-(7-chloro-2-quinolinyl)-ethenyl)phenyl)-3-(hydroxypropyl)phenyl)-2-propanol methanesulfonate described above is very unstable, especially if isolated in the solid form. At temperatures above −10° C. and in the presence of the diisopropylethylamine hydrochloride (byproduct), the resulting methanesulfonate undergoes irreversible intramolecular substitution which yields a cyclic ether. Furthermore, the methanesulfonate must be filtered and stored at very low temperatures, which restricts the application of the crystalline methanesulfonate in the industrial scale.

Therefore, it proved advantageous to develop a Montelukast synthesis method in which the crystalline methanesulfonate does not have to be isolated, which is the subject matter of the present invention.

### BRIEF SUMMARY OF THE INVENTION

The present invention provides a new method for the preparation of sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid:

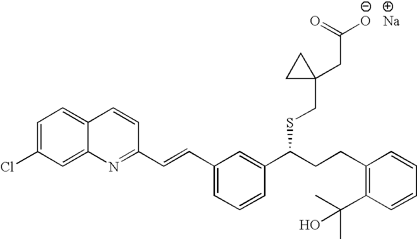

wherein crystalline forms of methanesulfonate and Montelukast-acid are not isolated, which ensures that the reaction proceeds with high yield and a product with high pharmaceutical purity is obtained.

The object of the invention was accomplished through the preparation of a tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid

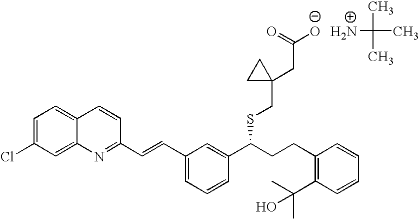

(disclosed in WO 2006/043846), its purification to achieve a product of pharmaceutical purity, and subsequent conversion into an amorphous form of Montelukast sodium salt.

The following crystalline intermediates are not isolated in the embodiments of this invention: 2-(2-(3(S)-(3-(2-(7-chloro-2-quinolinyl)-ethenyl)phenyl)-3-(hydroxypropyl)phenyl)-2-propanol methanesulfonate (methanesulfonate):

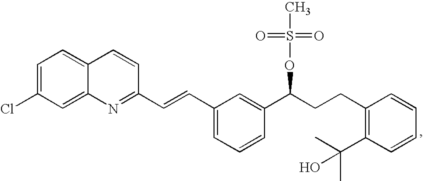

and/or the free 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid:

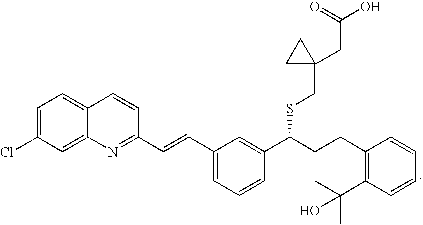

The method according to the present invention comprises the process for the preparation of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid sodium salt involving the reaction of 2-(2-(3(S)-(3-(2-(7-chloro-2-quinolinyl)-ethenyl)phenyl)-3-(hydroxypropyl)phenyl)-2-propanol:

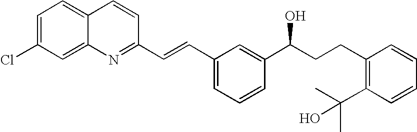

with methanesulfonyl chloride in the presence of a suitable tertiary amine which forms a hydrochloride that is insoluble in the reaction solvent. Appropriate solvents such as N,N-dimethylformamide, N,N-dimethylacetamide, dimethyl sulfoxide, tetrahydrofurane and N-methylpyrrolidone are used.

After mesylation, the mixture is filtered to separate the tertiary amine hydrochloride (byproduct), and the filtrate, crude 2-(2-(3(S)-(3-(2-(7-chloro-2-quinolinyl)-ethenyl)phenyl)-3-(hydroxypropyl)phenyl)-2-propanol methanesulfonate solution, is added to the previously prepared (1-(sulfanylmethyl)cyclopropyl)acetic acid disodium salt:

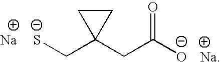

Thus, the possibility of decomposition of the methanesulfonate during the isolation is eliminated, and the yield of Montelukast synthesis remains comparable to those disclosed in the aforementioned patent applications.

Subsequently, 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid solution is isolated from the reaction mixture. From the solution, a crystalline tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid:

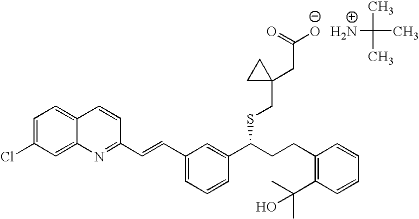

is isolated. The resulting salt is purified until a salt with high pharmaceutical purity is obtained. The tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is subsequently converted into the amorphous form of a sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid. Triethylamine is used as the tertiary amine.

The method for preparing tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid according to the present invention comprises reacting 2-(2-(3(S)-(3-(2-(7-chloro-2-quinolinyl)-ethenyl)phenyl)-3-(hydroxypropyl)phenyl)-2-propanol with methanesulfonate chloride in the presence of a tertiary amine. From the resulting crude 2-(2-(3(S)-(3-(2-(7-chloro-2-quinolinyl)-ethenyl)phenyl)-3-(hydroxypropyl)phenyl)-2-propanol methanesulfonate solution the precipitated amine hydrochloride is filtered off. Thereafter, the solution is directly reacted with (1-(sulfanylmethyl)cyclopropyl)acetic acid disodium salt of Formula 3. From the resulting reaction mixture, 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is isolated from which a crystalline tert-butylamine salt of 1-(((1(R)-(3-(2-(7-chloro-2-quinolinyl)-ethenyl)phenyl)-3-(2-(1-hydroxy-1-methylethyl)phenyl)propyl)-sulfanyl)methyl)cyclopropaneacetic acid is subsequently isolated. The salt is purified to a high pharmaceutical purity. Triethylamine is used as the tertiary amine.

The method for purifying a tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid according to the present invention comprises converting the tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid into a solution of free 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid using an organic acid in a toluene/methanol/water system; discarding the methanol/water phase, which contains some of the impurities; reacting the solution of free 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid with a tert-butylamine toluene solution; and crystallizing a tert-butylamine salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid.

The method for the preparation of a pharmaceutically acceptable salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid according to the present invention comprises reacting 2-(2-(3(S)-(3-(2-(7-chloro-2-quinolinyl)-ethenyl)phenyl)-3-(hydroxypropyl)phenyl)-2-propanol with methanesulfonyl chloride in the presence of a tertiary amine. From the resulting crude 2-(2-(3(S)-(3-(2-(7-chloro-2-quinolinyl)-ethenyl)phenyl)-3-(hydroxypropyl)phenyl)-2-propanol methanesulfonate solution the precipitated amine hydrochloride is filtered off, and the solution is directly reacted with (1-(sulfanylmethyl)cyclopropyl)acetic acid disodium salt of Formula 3. From the resulting reaction mixture 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is isolated, from which the crystalline form of a pharmaceutically acceptable salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is isolated. The salt is purified until high pharmaceutical purity is obtained. Triethylamine is used as the tertiary amine.

Prior art methods for the isolation of crystalline 1-(((1(R)-(3-(2-(7-chloro-2-quinolinyl)-ethenyl)phenyl)-3-(2-(1-hydroxy-1-methylethyl)-phenyl)propyl)sulfanyl)methyl)-cyclopropaneacetic acid and its crystalline sodium salt involve the preparation of a high purity tert-butylamine salt thereof. In prior art, the tert-butylamine salt is recrystallized from solvents, such as toluene, toluene/acetonitrile, toluene/isopropanol, toluene/ethanol, acetonitrile/water or ethyl acetate. However, the recrystallization of the salt not always ensures that impurities are efficiently removed and brought to an adequate level. Efficient removal of impurities has been accomplished herein by converting the Montelukast tert-butylamine salt into the free acid using acetic acid in toluene/methanol/water system. The methanol/water phase, which contains some of the impurities, is discarded and the solution of Montelukast free acid in toluene is treated with tert-butylamine and crystallized again as the tert-butylamine salt. The addition of a lower alcohol yields higher purity of the salt formed after crystallization. The above purification procedure is repeated until the salt purity is satisfactory. Purification yield is close to 90%.

1-(Mercaptomethyl)cyclopropaneacetic acid disodium salt of Formula 3 is obtained in the direct reaction between 1-(mercaptomethyl)cyclopropaneacetic acid and sodium tert-butanolate in the molar ratio of 1:2. As the solvent selected is N,N-dimethylformamide containing no more than 0.1% water. The same solvent is used in the preparation of methanesulfonate and Montelukast synthesis. Other appropriate solvents include N,N-dimethylformamide, dimethyl sulfoxide, tetrahydrofurane and N-methylpyrrolidone. The same solvent is preferably used both in the mesylation stage and in the coupling of mesylate with mercaptide. To the sodium tert-butanolate solution in N,N-dimethylformamide(1-(sulfanylmethyl)cyclopropyl)acetic acid (solid or in N,N-dimethylformamide solution) is added under nitrogen flow. Exothermic effect of the reaction is observed and the mixture becomes denser. Vigorous mixing of the reaction mixture ensures that the reaction proceeds completely. It was found that the mixing time needs to be extended for larger-scale processes.

The methanesulfonate of Formula 2 is formed in the reaction of a diol with methanesulfonyl chloride in N,N-dimethylformamide. Mesyl chloride is added dropwise in the presence of triethylamine while the temperature is maintained at between −20 and −15° C. The side product, triethylamine hydrochloride, is separated using a pressure filter, and the cooled filtrate (mesylate solution in N,N-dimethylformamide) is added straight to the previously prepared solution of 1-(mercaptomethyl)cyclopropaneacetic acid disodium salt (mercaptide).

The coupling of merged solutions of the intermediates (mercaptide with methanesulfonate) proceeds at temperatures between 0 and 25° C. under inert gas flow. For the reaction conducted at a temperature of 0-5° C., complete conversion of the methanesulfonate was observed after 20-25 hours; at a temperature of 10-15° C., the reaction time was 10-12 hours; and at a temperature of 20-25° C., the reaction was completed after only 2-3 hours. It is recommended for the reaction to proceed at lower temperatures; the higher the temperature, the greater tendency for the formation of unfavorable side products, especially after reaction completion.

Reaction progress was checked using high-performance liquid chromatography. The molar ratio of 1-(sulfanylmethyl)cyclopropyl)acetic acid to the diol was tested in the range between approximately 0.90:1 to approximately 1.55:1. Lower amounts of 1-(sulfanylmethyl)cyclopropaneacetic acid resulted in reduced yields. Higher molar excess of the acid ensures that the methanesulfonate is completely converted irrespective of the yield of the mesylation stage with greater risk of the formation of unfavorably high concentrations of impurities and reduced process yield. The most favorable molar ratio found was between 1.10:1 and 1.20:1.

After the reaction is completed, the mixture is neutralized using citric acid in water/toluene system. The isolated Montelukast solution in toluene is additionally washed with water. The product is isolated from the solution as a crystalline tert-butylamine salt. To this end the solution is treated with tert-butylamine and concentrated partially under reduced pressure to remove any residual water and excess of tert-butylamine.

To facilitate crystallization the concentrated product solution is seeded with crystals of pure Montelukast tert-butylamine salt. Crystallization time after seeding necessary for complete crystallization of the salt is at least 24 hours.

The amorphous form of Montelukast sodium is obtained by adding a concentrated Montelukast sodium solution in dichloromethane or toluene dropwise to a non-polar solvent, such as pentane or hexane. The tert-butylamine in the salt molecule is removed by extraction from water using acetic acid or directly using sodium hydroxide solution in methanol.

The high pharmaceutical purity of the amorphous form of sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is achieved in the reaction which does not involve the isolation of crystalline Montelukast sodium.

### BRIEF DESCRIPTION OF THE DRAWINGS

The objects and advantages of the present invention will become more readily apparent after reading the ensuing description of the non-limiting illustrative embodiments and viewing the accompanying drawings, in which

FIG. 1 shows the chemical structure of 2-(2-(3(S)-(3-(2-(7-chloro-2-quinolinyl)-ethenyl)phenyl)-3-(hydroxypropyl)phenyl)-2-propanol;

FIG. 2 shows the chemical structure of 2-(2-(3(S)-(3-(2-(7-chloro-2-quinolinyl)-ethenyl)phenyl)-3-(hydroxypropyl)phenyl)-2-propanol methanesulfonate;

FIG. 3 shows the chemical structure of sodium 2-(1-(sulfidomethyl)cyclopropyl)acetate;

FIG. 4 shows the chemical structure of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid tert-butylamine salt;

FIG. 5 shows the chemical structure of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid sodium salt;

FIG. 6 shows an X-ray powder diffraction pattern of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid tertbutylamine salt;

FIG. 7 shows an X-ray powder diffraction pattern of purified 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid tertbutylamine salt; and

FIG. 8 shows an X-ray powder diffraction pattern of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid sodium salt.

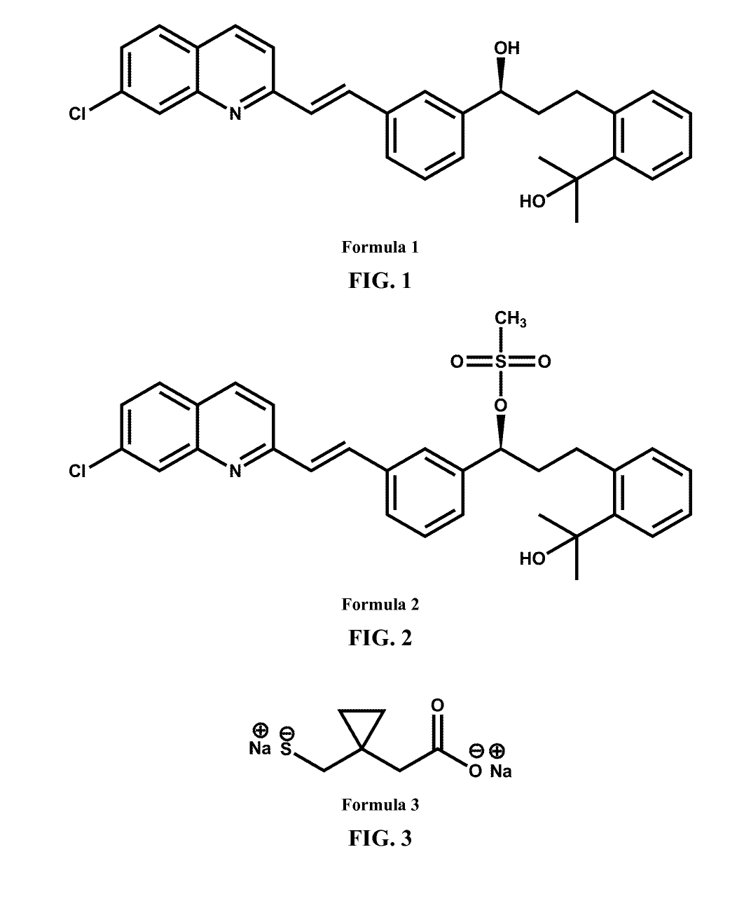

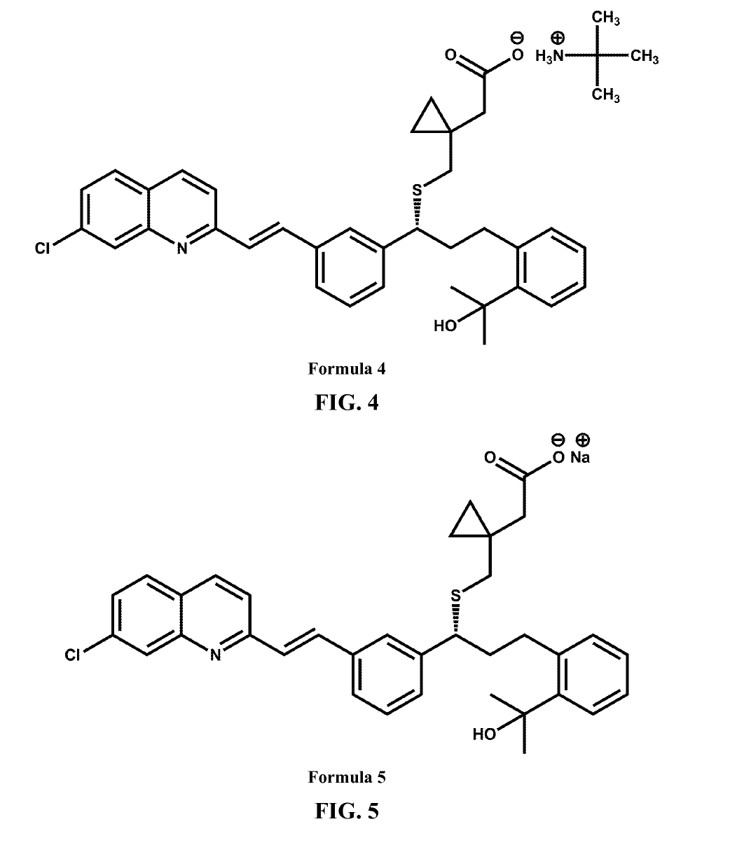

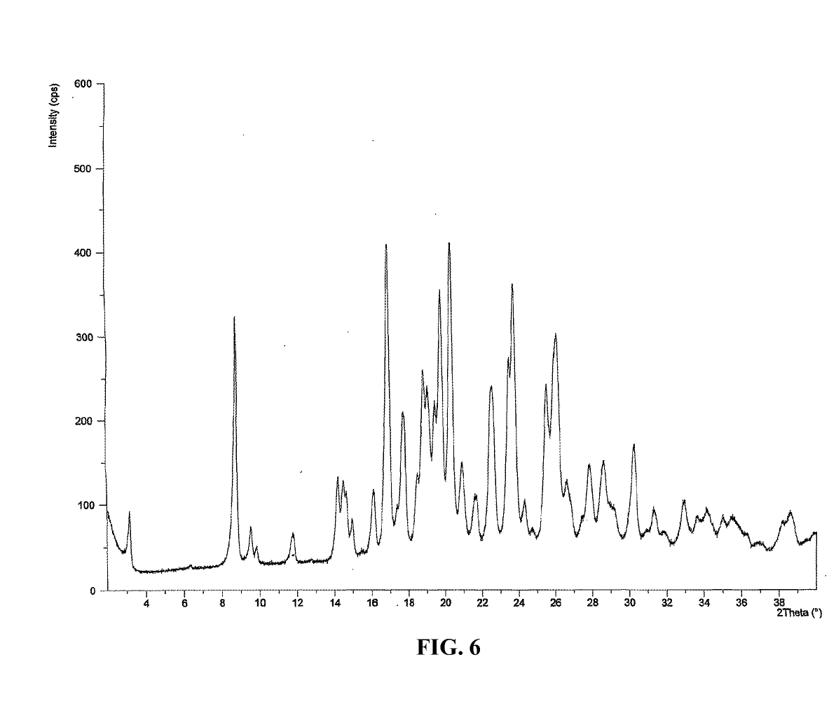

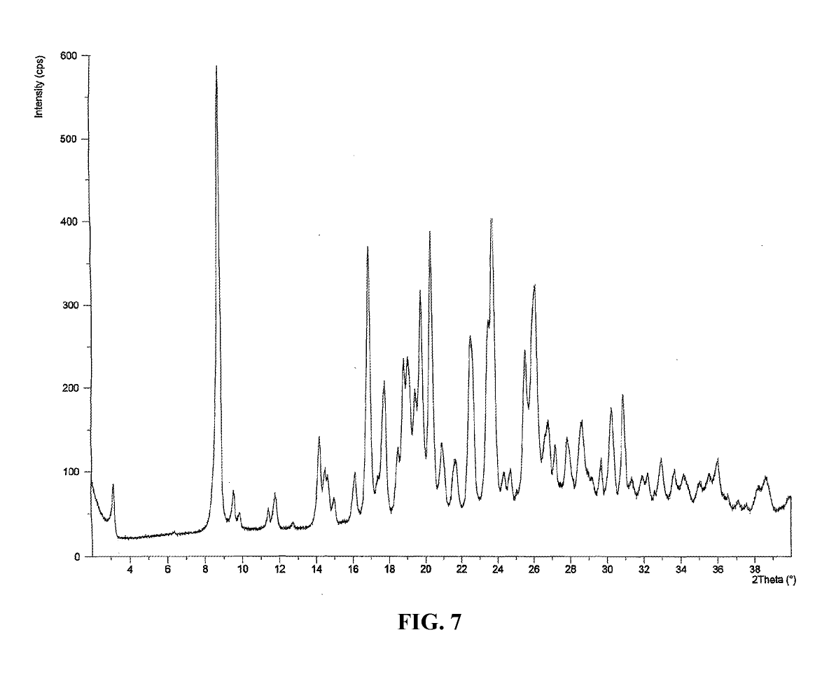

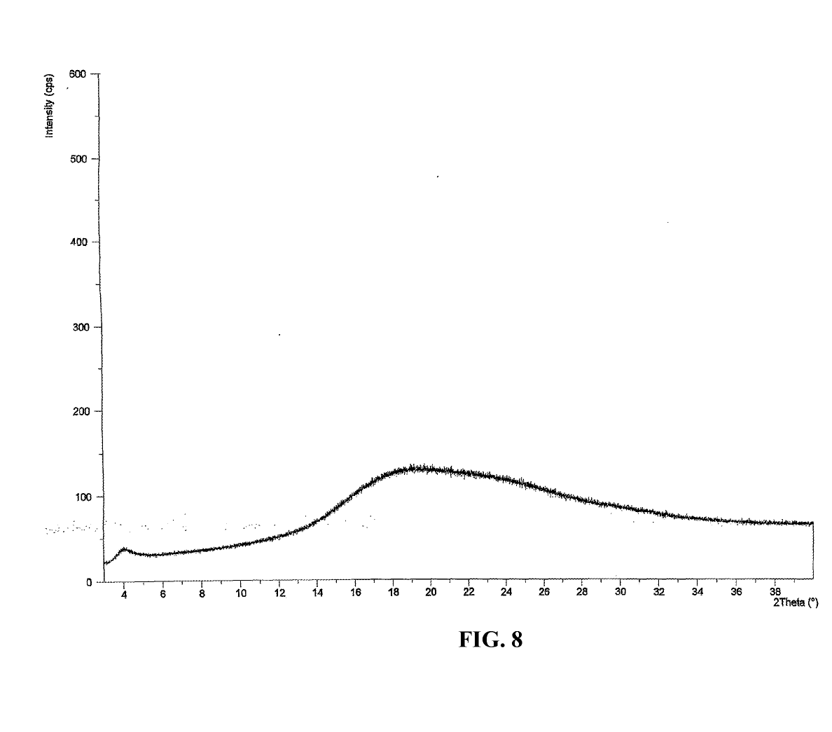

### DETAILED DESCRIPTION OF THE INVENTION

The following examples serve to illustrate the invention only and therefore should not be taken to limit the scope thereof.

### Example I

### Preparation of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid tert-butylamine salt

Stage 1.

A 1000 mL glass reactor fitted with a mechanical stirrer, thermometer and nitrogen inlet was flushed with nitrogen. 570 mL of N,N-dimethylformamide and 23.31 g (0.2426 mol) of sodium tert-butanolate was added under nitrogen flow and the content was stirred at 20±5° C. until the salt dissolved completely. 17.28 g (0.1182 mol) of solid 1-(sulfanylmethyl)cyclopropaneacetic acid was added to the resulting solution with vigorous stirring (exothermic reaction, temperature increase of approx. 10° C.). The resulting suspension with a jelly-like consistency was stirred vigorously under nitrogen flow for at least one hour at >15° C. The content was then cooled to 10-15° C.

Stage 2.

A 250 mL three-necked flask fitted with a stirrer, thermometer and dropping funnel was flushed with nitrogen. 45 g (0.0983 mol) of 2-(2-(3(S)-(3-(2-(7-chloro-2-quinolinyl)-ethenyl)phenyl)-3-(hydroxypropyl)phenyl)-2-propanol and 120 mL of N,N-dimethylformamide were poured into the flask and stirred until the diol dissolved completely. The content was cooled to between −20 and −15° C. in the CO₂/acetone bath. After cooling, 15.7 mL (11.43 g; 0.1130 mol) of triethylamine were added, the temperature was maintained at −20 to −15° C., and 12.38 g (8.4 mL; 0.1081 mol) of methanesulfonyl chloride were added dropwise over 40 minutes.

Stage 3.

After the addition of mesyl chloride was completed, the reaction mixture from Stage 2 (mesylate solution) cooled to −20 to −15° C. was added through a glass pressure filter to a reactor containing the suspension of 1-(mercaptomethyl)cyclopropaneacetic acid disodium salt (prepared in Stage 1) using compressed nitrogen. The triethylamine hydrochloride precipitate retained on the filter was washed with 30 mL of N,N-dimethylformamide. The washings were merged with the resulting reaction mixture.

The reaction mixture was maintained under nitrogen flow at 10-15° for 12 hours. The mixture was thereafter transferred to a 2500 mL glass reactor fitted with a mechanical stirrer and nitrogen inlet. 750 mL of toluene cooled to 0-5° C. were added and 750 mL of water cooled to 5° C. were added dropwise over one hour while maintaining the reactor temperature at 5-10° C. Subsequently, a solution of 7.2 g (0.0375 mol) of anhydrous citric acid in 76.5 mL of water was added dropwise over 15 minutes at 5-10° C. The content was stirred for 15 minutes and left to enable phase separation. The aqueous phase was discarded. The organic phase containing the product was washed with 375 mL of water and then washed with 375 mL of water and 76.5 mL of methanol at 5-10° C. 16.8 mL (11.71 g; 0.1601 mol) of tert-butylamine were added to the organic phase and stirred under nitrogen flow for 30 minutes The solution was then concentrated under reduced pressure at 35° C. About 300 mL of the solvent was distilled off including the excess of tert-butylamine. 5.4 mL of isopropanol was added to the solution after distillation along with a seed crystal of the aforementioned Montelukast tert-butylamine salt. The content was stirred under nitrogen flow at 20-25° C. for 22 hours, cooled to 0-5° C. and stirred at that temperature for 24 hours. The precipitate that formed was filtered using a pressure filter with compressed nitrogen and washed three times with 30 mL portions of cool toluene. The product was dried at 35° C. under reduced pressure. The yield was 35.5 g of crude tert-butylamine salt (93.6% purity as HPLC tested).

Stage 4.

35 g of the tert-butylamine salt (93.6% purity as HPLC tested) was poured into a 1000 mL flask fitted with a stirrer, thermometer, cooler and nitrogen inlet. 175 mL of methanol was added and the content was stirred until the salt dissolved completely. 350 mL of toluene, 2.12 mL of concentrated acetic acid and 175 mL of water were added to the solution. The content is stirred for 30 minutes and left to enable phase separation. The aqueous phase was discarded. 175 mL of toluene and 8.4 mL of tert-butylamine were added to the organic phase and the content was stirred for 20 minutes. Approx. 158 mL of the solvent was distilled off under reduced pressure from the resulting solution at 35° C.

175 mL of methanol, 2.12 mL of concentrated acetic acid and 175 mL of water were added to the solution after distillation. The content was stirred for 30 minutes and left to enable phase separation. The aqueous phase was discarded. 175 mL of toluene and 8.4 mL of tert-butylamine were added to the organic phase and the content was stirred for 20 minutes. Approx. 158 mL of the solvent was distilled off under reduced pressure from the resulting solution at 35° C.

17.5 ml, of isopropanol was added to the solution after distillation along with a crystal seed of Montelukast tert-butylamine salt. The content was stirred under nitrogen flow at 20-25° for 24 hours. The precipitate that formed was filtered using a pressure filter with compressed nitrogen and washed twice with 35 mL portions of toluene. The filtered salt was dried at 35° C. under reduced pressure. The yield was 29.7 g of the salt (98.2% purity as HPLC tested).

¹H NMR (500 MHz, CDCl₃): δ 8.11 (d, 1H, J=8.5 Hz), 8.07 (d, 1H, J=1.7 Hz), 7.73-7.65 (m, 4H), 7.50-7.09 (m, 9H), 3.99 (t, 1H, J=7.3 Hz), 3.22-3.15 (m, 1H), 2.91-2.84 (m, 1H), 2.58 (AB, 2H, J=12.9 Hz), 2.38 (AB, 2H, J=15.6 Hz), 2.30-2.13 (m, 2H), 1.62, 1.60 (2×s, 6H), 1.33 (s, 9H), 0.57-0.33 (m, 4H).

¹³C NMR (125.7 MHz, CDCl₃): 177.75, 156.89, 148.42, 145.30, 143.80, 140.28, 136.39, 136.23, 135.59, 135.25, 131.50, 128.91, 128.66, 128.53, 127.91, 127.12, 127.03, 126.66, 126.13, 125.61, 125.55, 125.37, 119.38, 73.60, 50.87, 50.14, 42.65, 40.02, 39.60, 32.26, 31.84, 28.46, 17.23, 12.80, 12.21.

To obtain high purity Montelukast tert-butylamine salt the purification step from Stage 4 was repeated.

The yield of a single purification stage was 80-90%.

### Example II

### Preparation of sodium 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetate

20.5 g of purified Montelukast tert-butylamine salt (99.7% purity as HPLC tested) was poured into a 500 mL flask fitted with a stirrer, thermometer, coller and nitrogen inlet (powder X-ray diffraction pattern shown in FIG. 2). Subsequently, 174 mL of dichloromethane and 1.78 mL of concentrated acetic acid were added. The resulting clear Montelukast solution in dichloromethane was washed twice with 102.5 mL water portions. The aqueous phases after washing were discarded and 13.29 mL of 2.34 M sodium hydroxide methanolic solution was added to the organic phase. 102.5 mL of the solvent was distilled off from the resulting solution under atmospheric pressure; thereafter, 82 mL of dichloromethane were added and 102.5 mL of the solvent were distilled off again. The mixture after distillation was filtered warm through filter paper. The filtrate was diluted with 31 mL of dichloromethane and added dropwise to 1025 mL of n-pentane at 15-20° C. The resulting suspension of the product was filtered using a pressure filter with compressed nitrogen. The filtered product was dried at 60-80° C. under reduced pressure. The yield was 17.3 g of amorphous Montelukast sodium (99.7% purity as HPLC tested, enantiomeric purity 99.96% as HPLC tested).

¹H NMR (500 MHz, CD₃OD): δ 8.26 (d, 1H, J=8.8 Hz), 7.97 (d, 1H, J=2.0 Hz), 7.88-7.70 (m, 4H), 7.57-7.05 (m, 9H), 4.91 (s, active H), 4.04 (dd, 1H, J=6.3 Hz), 3.10 (m, 1H), 2.83 (m, 1H), 2.65 (d, 1H, J=12.7 Hz), 2.52 (d, 1H, J=12.9 Hz), 2.40 (d, 1H, J=14.6 Hz), 2.30 (d, 1H, J=14.6 Hz), 2.29-2.10 (m, 2H), 1.53, 1.51 (2×s, 6H), 0.56-0.29 (m, 4H).

¹³C NMR (125.7 MHz, CD₃OD): 180.84, 158.76, 149.29, 147.05, 145.63, 141.31, 138.15, 137.69, 137.40, 136.81, 132.49, 130.48, 130.08, 130.03, 128.77, 128.33, 128.15, 127.90, 127.84, 127.25, 126.89, 126.48, 126.44, 120.81, 73.83, 51.42, 44.75, 41.32, 41.14, 33.47, 31.85, 18.53, 13.35, 12.80.

1-8. (canceled)

9. A method for the preparation of a sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, the method comprising:
a) reacting a compound of Formula 1 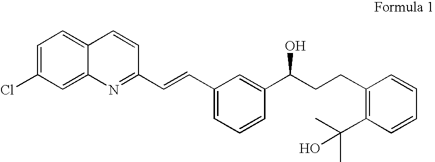
with methanesulfonyl chloride in the presence of a tertiary amine to yield a crude solution of a compound of Formula 2 and solid amine hydrochloride, 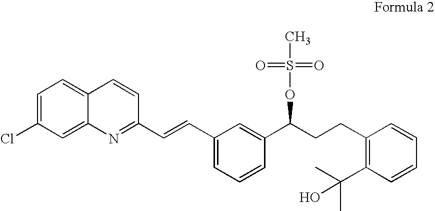
and filtering the crude solution to remove the solid amine hydrochloride and to obtain a filtrate comprising the compound of Formula 2;
b) reacting the filtrate comprising the compound of Formula 2 obtained in a) without isolation or further purification with a compound of Formula 3 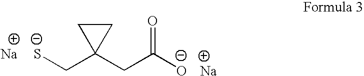
to yield 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, and isolating said 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid;
c) reacting the 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid isolated in b) with tert-butylamine to yield a compound of Formula 4; 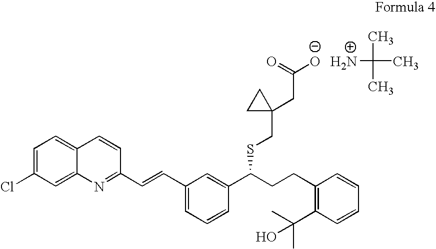
d) isolating and purifying the compound of Formula 4; and
e) converting the compound of Formula 4 to the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid.

10. The method of claim 9, wherein the tertiary amine is triethylamine.

11. The method of claim 9, wherein in (a) and (b), reactions are carried out in a solvent selected from N,N-dimethylformamide, N,N-dimethylacetamide, dimethyl sulfoxide, tetrahydrofurane or N-methylpyrrolidone.

12. The method of claim 9, wherein in d) purifying the compound of formula 4 comprises: reacting the compound of Formula 4 with an organic acid in a toluene/methanol/water system to yield a solution of free 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid; and discarding the methanol/water phase, treating the solution of free 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid with tert-butylamine in toluene, and isolating a crystalline solid of Formula 4.

13. The method of claim 9, wherein in e) converting the compound of Formula 4 to the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, comprises: reacting the isolated and purified compound of Formula 4 with a free acid to yield a solution of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, and subsequently adding sodium hydroxide to yield the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid.

14. The method of claim 9, wherein the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is amorphous.

15. A method for the preparation of a sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, the method comprising:
a) reacting a compound of Formula 1 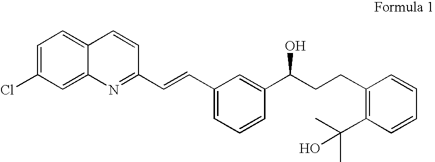
with methanesulfonyl chloride in the presence of a tertiary amine in a solvent to yield a solution of a compound of Formula 2; 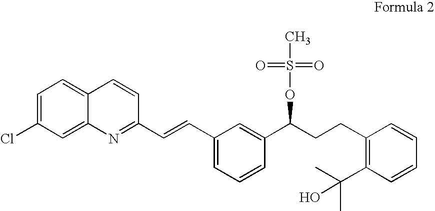
b) filtering the solution obtained in a), and without isolation or further purification, reacting the filtrate with a compound of Formula 3 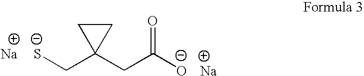
to yield 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid;
c) isolating said 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, and reacting the isolated 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid with tert-butylamine to yield a compound of Formula 4; 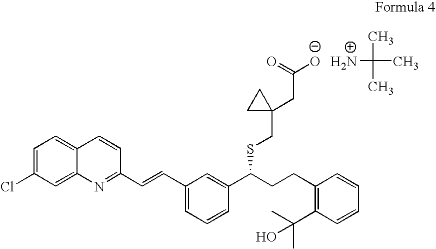
d) isolating and purifying the compound of Formula 4; and
e) converting the compound of Formula 4 to the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid.

16. The method of claim 15, wherein the tertiary amine is triethylamine.

17. The method of claim 15, wherein said solvent is selected from N,N-dimethylformamide, N,N-dimethylacetamide, dimethyl sulfoxide, tetrahydrofurane, or N-methylpyrrolidone.

18. The method of claim 15, wherein in d) purifying the compound of formula 4 comprises: reacting the compound of Formula 4 with an organic acid in a toluene/methanol/water system to yield a solution of free 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid; and discarding the methanol/water phase, treating the solution of free 1-(((1(R)-(3-(2-(7-chloro-2-quinolinyl)-ethenyl)phenyl)-3-(2-(1-hydroxy-1-methylethyl)phenyl)propyl)sulfanyl)methyl)-cyclopropaneacetic acid with tert-butylamine in toluene, and isolating a crystalline solid of Formula 4.

19. The method of claim 15, wherein in e) converting the compound of Formula 4 to the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, comprises: reacting the isolated and purified compound of Formula 4 with a free acid to yield a solution of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid, and subsequently adding sodium hydroxide to yield the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid.

20. The method of claim 15, wherein the sodium salt of 1-(((1(R)-(3-(2-(7-chloro-2-quinolinyl)-ethenyl)phenyl)-3-(2-(1-hydroxy-1-methylethyl)phenyl)propyl)sulfanyl)methyl)cyclopropaneacetic acid is amorphous.
